(19) United States
(12) Patent Application Publication
(10) Pub. No.: US 2019/0053938 A1
(43) Pub. Date: Feb. 21, 2019

(54) Portable, therapeutic electrical heating system

(71) Applicant: Sunbeam Products, Inc., Boca Raton, FL (US)

(72) Inventors: Lalit Mandal, Boca Raton, FL (US); Jill Walters, Delray Beach, FL (US)

(21) Appl. No.: 15/998,695

(22) Filed: Aug. 16, 2018

Related U.S. Application Data
(60) Provisional application No. 62/546,197, filed on Aug. 16, 2017.

(51) Int. Cl. A61F 7/00; H02J 7/00; H01R 13/72; A61F 7/02

(52) U.S. Cl. CPC A61F 7/007; H02J 7/0047; H01R 13/72; A61F 7/02; A61F 2007/0249; H02J 2007/005; A61F 2007/0226; A61F 2007/0027; A61F 2007/0078; H02J 2007/0049

(57) ABSTRACT

A portable electrical heating system includes a controller, a heat delivery mechanism that is releasably and reusably attachable directly to a user's skin, and an electrical connection between the two that allows for selectively engagement between the controller and heat delivery mechanism. The controller allows for the selection of a temperature setting for the heat delivery mechanism. The controller includes at least one signaling device to indicate the power and temperature setting. The heat delivery mechanism includes a heating element, and further includes an adhesive layer that allows for multiple uses of the heat delivery mechanism.

### CROSS-REFERENCE TO RELATED APPLICATIONS

This application claims the benefit of U.S. Provisional Application No. 62/546,197 entitled PORTABLE ELECTRICAL HEATING PATCH, filed Aug. 16, 2017, which is hereby incorporated by reference in its entirety.

### FIELD OF INVENTION

The invention relates generally to the delivery of therapeutic heat to a human user, and more particularly, to a portable electric heating system with a mechanism to deliver therapeutic heat that is affixable directly to the skin of a human user.

### BACKGROUND

Pain can be caused by muscle exertion or strain, which can creates tension in the muscles and soft tissues. The tension can cause circulation to constrict and send pain signals to the brain. Heating pads can ease the pain by dilating the blood vessels in the painful area to increase blood flow and provide the area with additional oxygen and nutrients to help the damaged muscle and tissue heal. Heating pads can also stimulate sensation in the skin to decrease the pain signals being transmitted to the brain, and also decrease stiffness in the soft tissues surrounding the injured area.

There are several types of heating pads, including electrical, chemical, and hot water bottles, some of which are portable. While there are other portable heating pads, none of them utilize any way to deliver heat through direct skin application. The impact of the portable heating pads is therefore suboptimal because the concentration and net transfer of heat to the human body is not as direct and as impactful. It is therefore desirable for a system that includes a component that can be worn, placed, and attached directly on the skin to provide the maximum amount of heat.

### SUMMARY OF INVENTION

According to a first embodiment, a portable, therapeutic electrical heat system is provided for providing direct and effective heat on various parts of the body of a human user. Embodiments of the electrical heating system use a battery within an electrically powered controller to provide selective and controllable electrical power to a heat delivery mechanism that may include one or more components, each such component having a heating element and adhesive layer suitable for releasably adhering the component to a skin surface of a human user and which can be reused multiple times. In one embodiment, the heating element includes a carbon fiber heating element, and the adhesive layer includes a silicone gel. An electrical connector connects the controller with each component of the heat delivery mechanism, and is further able to wrap around the controller to facilitate carrying of the system when in use.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

For a better understanding of the various embodiments of the present invention, reference may be made to the accompanying drawings in which:

FIG. 1 is a front perspective view of an electronic heating system;

FIG. 2 is an exploded perspective view of the electronic heating system of FIG. 1;

FIG. 3 is a front perspective view of a controller of FIGS. 1 and 2;

FIG. 4 is a front perspective view of the controller of FIGS. 1-3 and at least one electrical connector of FIGS. 1-2;

FIG. 5 is a rear perspective view of the controller of FIGS. 1-4;

FIG. 6 is a top plan view of a kidney bean shaped patch;

FIG. 7 is a top plan view of a rectangular shaped patch of FIG. 1.

While the disclosure is susceptible to various modifications and alternative forms, specific embodiments thereof are shown by way of example in the drawings and will herein be described in detail. It should be understood, however, that the drawings and detailed description presented herein are not intended to limit the disclosure to the particular embodiments disclosed, but on the contrary, the intention is to cover all modifications, equivalents, and alternatives falling within the spirit and scope of the present disclosure.

### DETAILED DESCRIPTION OF THE DRAWINGS

The present invention will now be described with reference to the drawing figures, in which like reference numerals refer to like parts throughout. For purposes of clarity in illustrating the characteristics of the present invention, proportional relationships of the elements have not necessarily been maintained in the drawing figures.

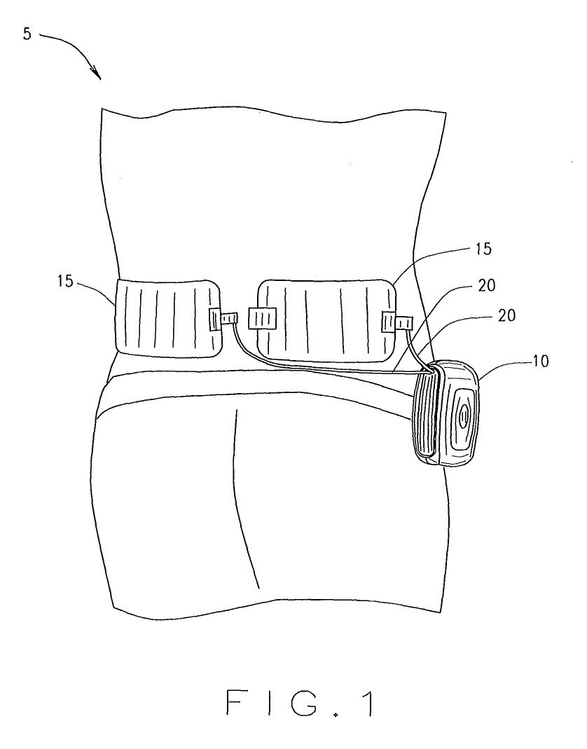

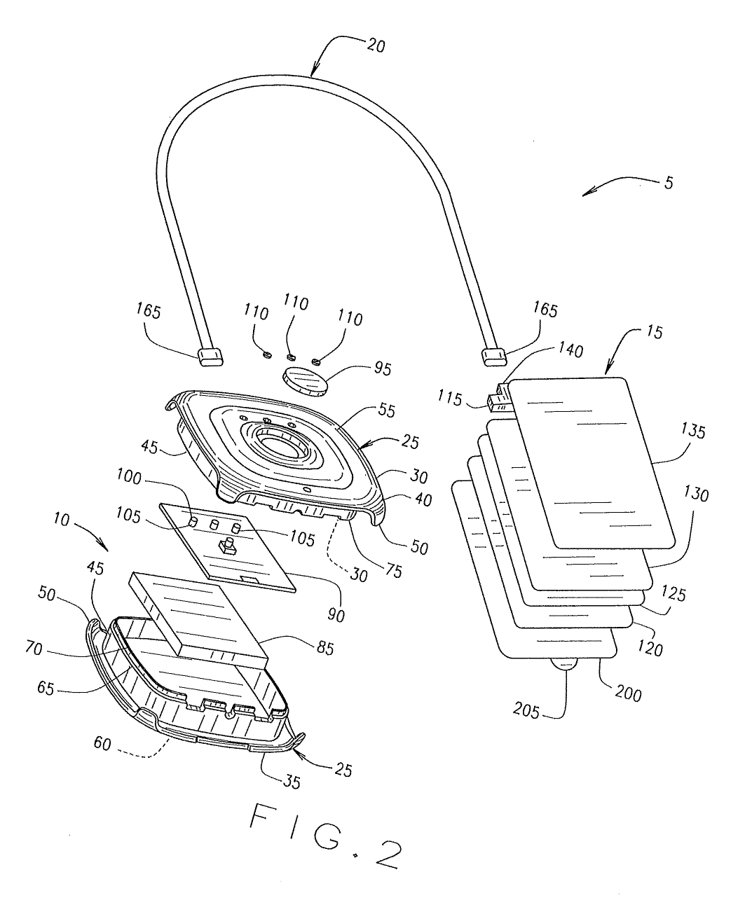

Turning to FIG. 1, the portable, therapeutic electrical heating system 5 includes an electrically powered controller 10, at least one heat delivery mechanism 15, which in one embodiment takes the form of a laminated, generally flat patch, and at least one electrical connector 20. In greater detail, and as illustrated in FIG. 2, the electronically powered controller 10 includes a housing 25, which includes an upper shell 30 and a bottom shell 35 that engage one another to create the housing 25. In one embodiment, both the upper shell 30 and bottom shell 35 have an outer section 40 and an inner section 45, and when the upper shell 30 and bottom shell 35 engage one another, the inner sections 45 of both the upper shell 30 and bottom shell 35 are adjacent and abut one another. When assembled, the housing 25 therefore has two outer sections 40 and one inner section 45. In one embodiment, both the outer sections 40 and the inner section 45 are generally substantially rectangular members, although these components may be provided in any number of other shapes without significantly affecting the functionality of the overall system. In this embodiment, the outer sections 40 have a larger perimeter than the perimeter of the inner section 45. The housing 25 therefore preferably has a spool-like shape. The outer sections 40 may further have at least one securing section 50 that extends to secure the electrical connector 20. In one embodiment, the at least one securing section 50 includes the corners of the outer section 40 that are further curved or bent inwardly, towards the inner section 45. The at least one electrical connector 20 may therefore wrap around the inner section 45 of the housing 25 and be secured by the securing section 50 of the outer sections 40, as will be explained in greater detail hereinafter. However, in alternative embodiments, the housing 25 can be any shape and size as long as it is able to surround the components of the controller 10 and can secure the at least one electrical connector 20, which will be explained in greater detail hereinafter.

The upper shell 30 of the housing 25 is preferably the top face or outer face 55, while the bottom shell 35 is preferably the bottom face or the inner face 60, when the electrical heating system 5 is carried using a belt clip 170. In other words, when the electrical heating system 5 is worn by the human user, the bottom shell 35 is adjacent to the user and is generally not visible. The upper shell 30, on the other hand, is opposite from the bottom shell 35 and is visible to the user when the electrical heating system 5 is worn. In one embodiment, the bottom shell 35 further includes a bottom rim 65 and a lower rim 70, with a smaller perimeter than the perimeter of the bottom rim 65, and projects upwardly from the bottom rim 65 towards the upper shell 30 when the upper shell 30 and bottom shell 35 engage one another. Similarly, the upper shell 30 may also include an upper rim 75, where an upper lip 80 is set within the upper rim 75 to create a step within the upper shell 30, and the upper lip 80 further having a smaller perimeter than the perimeter of the upper rim 75. When the upper shell 30 engages with the bottom shell 35, the upper shell 30 is positioned over the bottom shell 35 where the bottom shell 35 is inserted into the upper shell 30, so that the bottom rim 65 of the bottom shell 35 and the upper rim 75 of the upper shell 30 abut and rest upon each other, while the lower rim 70 abuts and rests upon the upper lip 80. The upper shell 30 and bottom shell 35 therefore engage one another through a friction or press fit or in other embodiments may include a lip and at least one recess elements on the respective shells that are operable to engage one other in an interference fit to removably secure the two shells together. Other methods of engaging the upper shell 30 and bottom shell 35 are envisioned and foreseeable. The housing 25 is preferably made out of plastic, but can be any semi-rigid or rigid material instead.

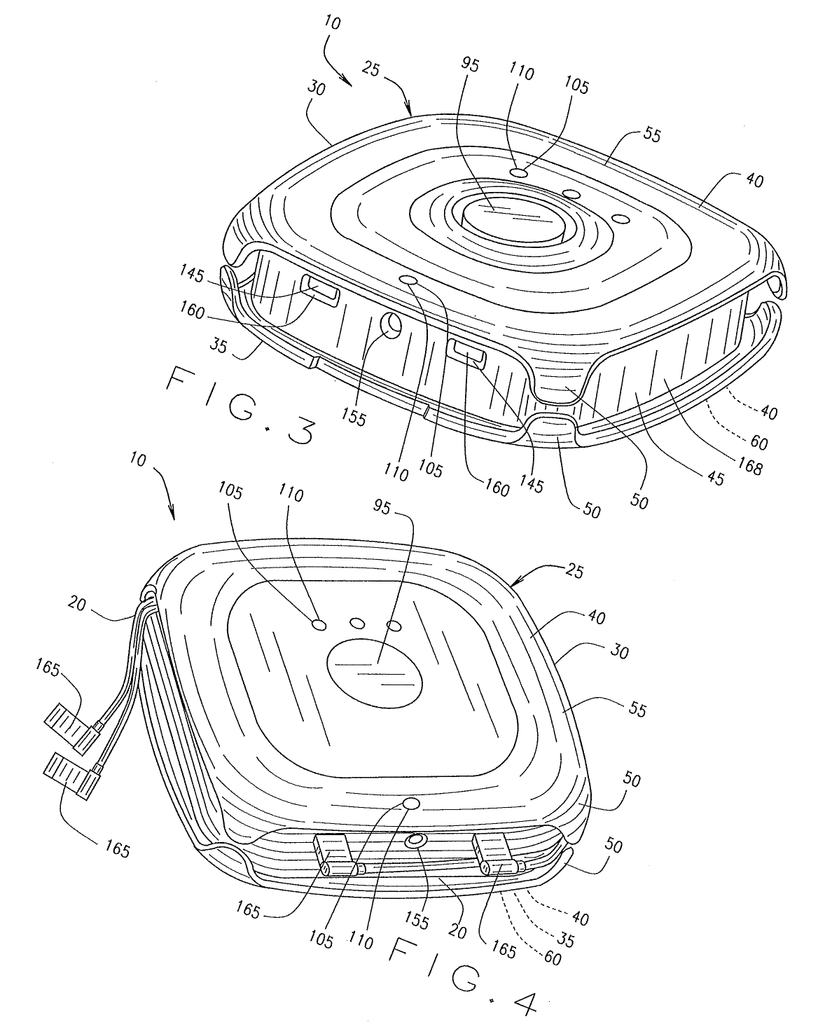

The housing 25 of the controller 10 preferably serves to protect and contain the components of the controller 10. The components of the electrically powered controller 10 include an electrical power source 85, which in one embodiment is a battery or battery pack, and may further which preferably includes one or more lithium ion batteries. The electrical power source 85 provides power to a first printed circuit board 90, connected to a temperature selection mechanism 95, which may be an on/off button or switch. In a particularly preferred embodiment, the electrical power source 85 is a rechargeable battery to reduce waste. The temperature selection mechanism 95 is preferably located and positioned on the outer face 55 of the housing 25, where the human user can push the temperature selection mechanism 95 to turn the power on or off, as seen in FIGS. 2 and 3. The temperature selection mechanism 95 can further be pushed to adjust the temperature setting of the electrical heating system 5 until the desired setting of low, medium, and high heat has been reached.

The controller 10 also includes at least one indicator 100 reflecting at least one operation state of the electric heating system 5 (e.g., the at least one operation variable may be when the controller 10 is on, the current temperature setting, or when the electrical power source 85 needs to be recharged). Thus, the controller may include a least one battery status indicator, an on/off indicator, and a temperature setting indicator. In one embodiment, the signaling device 100 of the controller 10 may be visual indicators in the form of a series of LED lights 105 that preferably will indicate whether the controller 10 has power, if it needs to be recharged, and its temperature setting. In one embodiment, the series of three LED lights 105 may turn on or change different colors, notifying the user of the selected pre-determined temperature setting. In the same or another embodiment, another LED light 105 may turn on or change different colors, notifying the user if the electrical power source 85 is fully charged, has a low charge, or if the electrical power source 85 has any charge remaining. The LED lights 105 may be covered and encased within at least one lens 110 to protect the LED lights 105 from being damaged, as can be seen in FIGS. 2-4. The lens 110 may be made out of plastic, but can be any material that is at least somewhat transparent so the user can see whether the LED light 105 is lit or the color the LED light 105.

As seen in FIG. 2, the first printed circuit board 90 and electrical power source 85 within the housing 25 is further connected to at least one electrical connector 20 to provide instructions and power to a second printed circuit board 115 within the at least one heat delivery mechanism 15, which will be explained in more detail hereinafter. In one embodiment, the at least one heat delivery mechanism 15 is a disposable patch that may be used multiple times, and preferably includes an adhesive layer 120, a first insulation layer 125, a heating panel layer 130, and a second insulation layer 135. The adhesive layer 120 is placed adjacent against the human user's skin, thereby allowing the heat delivery mechanism 15 to attach and remain attached to the user's skin. In other words, the adhesive layer 120 allows the heat delivery mechanism 15 to operably releasably secure the heat delivery mechanism to the skin surface of a user. The heat delivery mechanism 15 may be used repeatedly multiple times, in some cases as many as ten or more times, before the heat delivery mechanism 15 must be replaced due to the adhesive layer 120 losing its adhering properties due to the chemical adhesion of the adhesive layer 120 weakening over repeated uses. The adhesive layer 120 may be a silicone gel, although the adhesive layer 120 may be other materials, as long as the adhesive layer 120 allows the at least one heat delivery mechanism 15 to attach to the skin of the human user.

The heat delivery mechanism 15 includes the first insulation layer 125, which may be cotton fabric, and is positioned adjacent to and between the adhesive layer 120 and the heating panel layer 130. The heating panel layer 130 is preferably polyurethane and further contains a heating element for generating heat. In one embodiment, the heating element may be carbon fibers. The carbon fibers may be woven into the polyurethane and can carry low currents of electricity which allows the heat delivery mechanism 15 to heat to the desired pre-selected temperature. The flexibility of the carbon fibers further allows the heat delivery mechanism 15 to conform to different parts of the human user's body to better deliver a more consistent and direct transfer of heat and can sustain higher temperatures than, in particular, chemical heating pads. These carbon fiber heating elements are capable of maintaining temperatures in excess of 100° C., although in the current application temperatures that high are not generally therapeutically necessary. The heating panel layer 130 is located and positioned adjacent and in between both the first insulation layer 125 and the second insulation layer 135. The second insulation layer 135 is further located adjacent to a garment (if worn) when the heat delivery mechanism 15 is placed and attached to the skin of the user. The second insulation layer 135 may be polyester fabric, and prevents heating panel layer 130 from directly transferring heat to the garment or into the air. Each of these layers is preferably substantially the same shape and size. Finally, the heat delivery mechanism 15 also includes a second printed circuit board 115 which includes female connecting pins 140, allowing at least one male electric connection head 165 of the at least one electrical connector 20 to be inserted into and mated with the female connecting pins 140, therefore allowing the second printed circuit board 115 to be in electrical communication with the first printed circuit board 90 of the controller 10, which will be explained in more detail hereinafter.

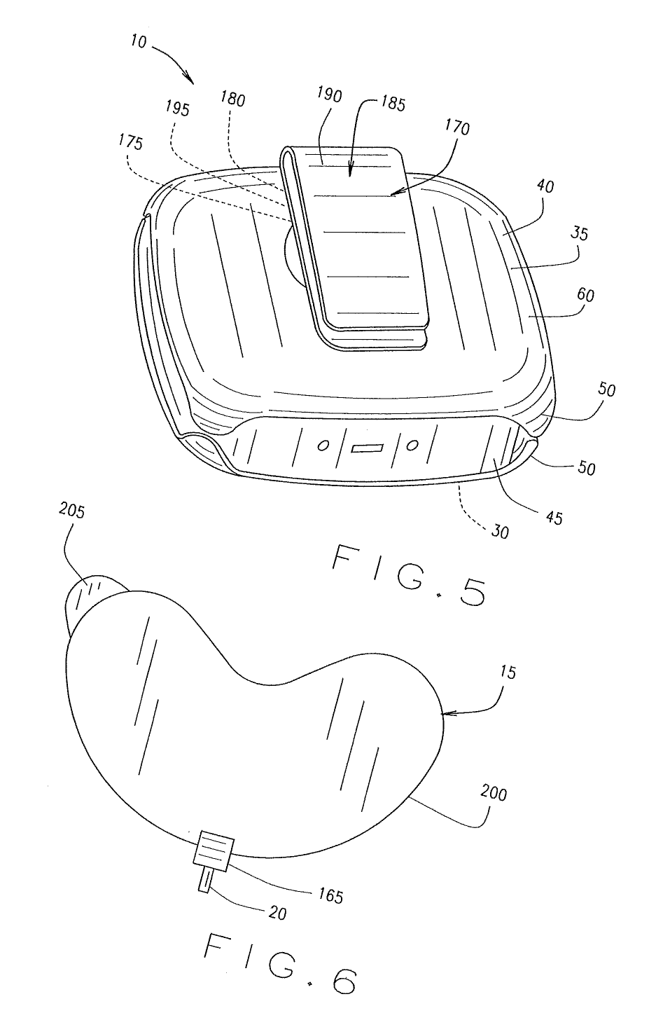

As illustrated in FIGS. 3-5, the controller 10 further includes at least one female connector 145 capable of mating with at least one male plug 150. In one embodiment, the controller 10 preferably includes a female pinhead connector 155 that mates with a male pinhead plug connected to a cable and a cable plug, which is capable of mating with a wall outlet, therefore allowing the electrical power source 85 of the controller 10 to recharge. Therefore, when the electrical power source 85 of the controller 10 needs to be recharged, the human user may insert the male pinhead plug into the female pinhead connector 155 and plug the cable plug into the wall outlet to recharge the electrical power source 85. The controller 10 may further include at least one female flat connector 160 capable of mating with the male flat electric connection head 165 at both ends of each at least one electrical connector 20. The male flat electric connection head 165 at one end of the electrical connector 20 can be inserted into the female flat connector 160 in the controller 10, while the male flat electronic connection head 165 at the other end of the electronic connection 20 can be inserted into the female connecting pins 140 in the second printed circuit board 115 of the at least one heat delivery mechanism 15, therefore allowing the controller 10 to be in electronical communication with the at least one heat delivery mechanism 15. In other words, the first printed circuit board 90 of the controller 10 is able to communicate with the second printed circuit board 115 of the heat delivery mechanism 15.

Once the male flat electric communication head 165 has been inserted into the female flat connector 160 of the controller 10, the at least one electrical connector 20 may be wrapped around the inner section 45 of controller 10 to secure and hold the at least one electrical connector 20 out of the way, but still allows for easy access if the length of the at least one electronic connection 20 needs to be adjusted. In other words, the inner section 45 and outer section 40 of the controller engage and coordinate one another to define a recess 168 around the periphery of the controller where the at least one electrical connector 20 may be selectively wrapped around the inner section 45 of the housing 25. The outer sections 40 each further have at least one securing section 50 that extends to coordinate the securing of the electrical connection 20 around the recess or the inner section 45, and prevent the electrical connection from unraveling when wrapped within the recess 168.

The inner face 60 of the controller 10 further includes a selectively engageable belt clip 170 so that a user may clip the controller 10 to a pair of pants or shorts, and use the electrical heating system 5 without being forced to remain in one place. In greater detail, the inner face 60 of the controller 10 includes at least one female snap button connector 175 located and positioned on the inner face 60 of the controller 10 that selectively engages at least one male snap button fastener 180 located and positioned on a holder 185 of the belt clip 170. The holder 185 is preferably an upside down U-shaped member where a user may slide his or her waistband in between the arms of the U-shaped member so that the holder 185 may selectively engage the waistband of a pair of pants. The holder 185 further has an inner side 190 and an outer side 195, where the inner side 190 is adjacent and abuts the user's undergarments if the user is using the belt clip 170 to facilitate carrying the controller 10. The outer side 195 of the holder 185 is adjacent to the inner face 60 of the controller 10 and includes the at least one male snap button fastener 180.

The at least one male snap button fastener 180 and at least one female snap button connector 175 are conventional type cooperatively engaging fasteners well known in the prior art. In operation, force is typically applied to the prongs of the male snap button fastener 180 so that the prongs are inwardly depressed when inserted into the female snap button connector 175. Once pressure is no longer applied to the prongs of the male snap button fastener 180, the prongs return to their normal position within the female snap button connector 175 thereby securing the male snap button fastener 180 within the female snap button connector 175. In order to disengage male snap button fastener 180 from the female snap button connector 175, the user applies force by pulling the male snap button fastener 180 away from the female snap button connector 175, thereby inwardly depressing the prongs and withdrawing the male snap button fastener 180 from the female snap button connector 175. The male snap button fastener 180 is therefore able to selectively engage with the female snap button connector 175, and the belt clip 170 therefore is selectively attachable and releasable from the controller 10.

In order to use the electrical heating system 5, the user may first check the at least one signaling device 100 to see whether the electrical power source 85 in the controller 10 has power or if the electrical power source 85 needs to be recharged or replaced, depending on the embodiment. In one embodiment where the at least one signaling device 100 is a LED light 105, the LED light 105 can turn on, change colors, or otherwise signal that the electrical power source 85 has no or a low charge. If the electrical power source 85 needs to be recharged, the user may insert the cable plug into the wall socket and the male pinhead plug into the female pinhead connector 155 of the controller 10. The LED light 105 can further indicate that the electrical power source 85 is charging and when the electrical power source 85 is fully charged.

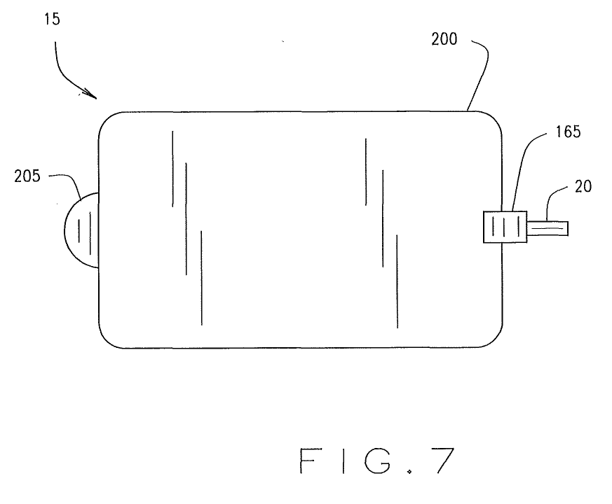

Once the electrical power source 85 has been sufficiently charged, the male pinhead plug may be removed from the female pinhead connector 155 of the controller 10, and the male flat electric connection head 165 of the at least one electrical connector 20 may be inserted into and mated to the at least one female flat connector 160 of the controller 10. The other male flat electric connection head 165 of the at least one electrical connector 20 may be inserted into the at least one heat delivery mechanism 15 so that the male flat electric connection head 165 mates with the female connecting pins 140. As seen in FIGS. 6 and 7, the heat delivery mechanism 15 is preferably rectangular or kidney-bean shaped. However, the heat delivery mechanism 15 may be any shape and size. The at least one heat delivery mechanism 15 also includes a plastic film 200 located and positioned over the adhesive layer 120, which should be removed prior to use, and further include a tab 205 to allow for easy removability of the plastic film 200. The plastic film 200 may be saved and re-positioned over the adhesive layer 120 of the at least one heat delivery mechanism 15 to prolong its adhering properties of the adhesive layer 120 when the at least one heat delivery mechanism 15 is stored for future use.

Once the plastic film 200 has been removed from the adhesive layer 120, the at least one heat delivery mechanism 15 may be placed again the human user's skin so that the adhesive layer 120 is adjacent and abuts the skin of the human user. Once the at least one heat delivery mechanism 15 has been positioned and attached at the desired area, the excess cable of the at least one electrical connector 20 may be wound around the inner section 45 of the controller 10 so that at least one electrical connector 20 does not get caught on other objects or become tangled. In an alternate embodiment, a split hard shell case is provided within which the heat delivery mechanism 15 can be stored between uses. In a preferred version of this embodiment, the inside surfaces of the case (those against which the heat delivery mechanism 15 may rest) are provided with multiple thin, raised fins that serve to minimize the surface contact between the shell and the heat delivery mechanism 15.

If the user desires to move while using the electrical heating system 5, the user may have the belt clip 170 engage with the controller 10 by inserting the male snap button fastener 180 into the female snap button connector 175 on the inner face 60 of the controller 10. The user can then use the belt clip 170 to facilitate carrying the controller 10, and therefore is not required to carry the controller 10 by hand. The user can then press the temperature selection mechanism 95 to turn the controller 10 on and further press the temperature selection mechanism 95 to adjust the temperate setting to a pre-determined setting, if so desired. The first printed circuit board 90 communicates the selected temperature through the at least one electrical connector 20 to the at least one heat delivery mechanism 15 by engaging the second printed circuit board 115 through the at least one electrical connector 20. The at least one electrical connector 20 is coupled to the first printed circuit board 90, which is further coupled to the electrical power source 85. The electrical connections extend from the controller 10 to the at least one heat delivery mechanism 15, where the electrical connectors 20 are may be inserted into the controller 10 and the at least one heat delivery mechanism 15. Thus, the electrical power source 85 is able to provide power to the at least one heat delivery mechanism 15, and the printed circuit boards 90 and 115 can communicate the desired temperature to the at least one heat delivery mechanism 15, so that the heat may be directed to the desired area on the user.

The various constructions described above and illustrated in the drawings are presented by way of example only and are not intended to limit the concepts and principles of the present invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present constructions and systems will, however, become apparent to those skilled in the art after considering the specification and the accompanying drawings. All such changes, modifications, variations and other uses and applications which do not depart from the spirit and scope of the invention are deemed to be covered by the invention.

What is claimed is:

1. A portable, therapeutic electrical heating system for a human user comprising:
An electrically powered controller including an electrical power source, a temperature selection mechanism and at least one indicator to reflect at least one operation state of the system;
at least one heat delivery mechanism, the heat delivery mechanism including a heating element for generating heat and an adhesive layer operable for releasably securing the heat delivery mechanism to a skin surface of the human user;
at least one electrical connector that is selectively engageable with the controller and further selectively engageable with the heat delivery mechanism and operable to enable electrical communication between the controller and the heat delivery mechanism; and
wherein the temperature selection mechanism is operable to turn the system on and off and to adjust the amount of heat delivered to the skin surface of the human user by the heat delivery mechanism.

2. The portable, therapeutic electrical heating system of claim 1, wherein the controller includes a housing having an outer section and an inner section;
the outer section and inner section coordinating to define a recess therebetween around the periphery of the controller within which the at least one electrical connector may be selectively wrapped around the inner section of the housing.

3. The portable, therapeutic electrical heating system of claim 1, further including a belt clip that is selectively attachable and releasable from the controller.

4. The portable, therapeutic electrical heating system of claim 1, wherein the temperature selection mechanism further includes an on/off button operable to selectively turn on and off a connection between the electrical power source and the controller.

5. The portable, therapeutic electrical heating system of claim 4, wherein the on/off button is further operable to select one of multiple, pre-set temperature settings for the heat delivery mechanism.

6. The portable, therapeutic electrical heating system of claim 1, wherein the electrical power source comprises at least one battery.

7. The portable, therapeutic electrical heating system of claim 6, wherein the at least one battery is rechargeable.

8. The portable, therapeutic electrical heating system of claim 1, wherein the controller includes at least one of a battery status indicator, an on/off indicator, and a temperature setting indicator.

9. The portable, therapeutic electrical heating system of claim 1, wherein the heat delivery mechanism comprises at least one patch.

10. The portable, therapeutic electrical heating system of claim 1, wherein the adhesive layer comprises a silicone gel.

11. The portable, therapeutic electrical heating system of claim 1, wherein the heat delivery mechanism further includes at least one insulation layer adjacent to a heating panel layer that contains the heating element.

12. The portable, therapeutic electrical heating system of claim 1, wherein the heating element comprises a carbon fiber heating element.

13. A portable, therapeutic electrical heating system for a human user comprising:
a controller including an electrical power source, a temperature selection mechanism, at least one indicator operable to provide a signal perceivable by the human user to indicate a power status of the system and a temperature setting of the system;
at least one heat delivery mechanism including an adhesive layer, at least a first insulation layer, and a heating panel layer containing at least one heating element operable for generating heat;
at least one electrical connector being selectively engageable with the controller and further selectively engageable with the at least one heat delivery mechanism to enable electrical communication between the controller and the heat delivery mechanism.

14. The portable, therapeutic electrical heating system of claim 13, wherein the adhesive layer includes a silicone gel, the first insulation layer includes one of a cotton fabric and a polyester fabric, and the heating panel layer includes polyurethane.

15. The portable, therapeutic electrical heating system of claim 13, wherein the heat delivery mechanism further includes at least a second insulation layer, the second insulation including one of a cotton fabric and a polyester fabric.

16. The portable, therapeutic electrical heating system of claim 13, wherein the heating panel layer includes a carbon fiber heating element.

17. The portable, therapeutic electrical heating system of claim 13, wherein the controller includes a housing having two outer sections and an inner section, the outer sections and inner section coordinating to form a recess around the periphery of the controller within which the electrical connection may be wound, the outer sections each further having at least one extending section, the extending sections of the outer sections coordinating to secure the electrical connection within the recess and prevent the electrical connection from unraveling when wrapped within the recess.

18. The portable, therapeutic electrical heating system of claim 13, further including a belt clip that is selectively attachable and releasable from the controller and is operable for facilitating carrying of the controller.

19. The portable, therapeutic electrical heating system of claim 13, wherein the electrical power source further includes a rechargeable battery.

20. A portable, therapeutic electrical heating system for a human user comprising:
a controller including a rechargeable electrical power source, a temperature selection mechanism, at least one indicator operable to provide a signal perceivable by the human user to indicate a power status of the system and a temperature setting of the system;
at least one heat delivery patch including a silicone gel adhesive layer operable for selectively and releasably securing the heat delivery patch to a skin surface of the human user, at least a first insulation layer, and a heating panel layer containing at least one carbon fiber heating element operable for generating heat;
at least one electrical connector being selectively and releasably engageable with the controller and further selectively and releasably engageable with the at least one heat delivery patch to enable electrical communication between the controller and the heat delivery patch.
